(19) United States
(12) Patent Application Publication
(10) Pub. No.: US 2012/0116377 A1
(43) Pub. Date: May 10, 2012

(54) APPARATUS UTILIZING A GUIDE WIRE TO MANEUVER HEATING ELEMENT AND PROBE FOR TREATING DAMAGED SPINAL DISCS

(76) Inventor: Elizabeth Ann Snoderly, Murrells Inlet, SC (US)

(21) Appl. No.: 13/351,387

(22) Filed: Jan. 17, 2012

Related U.S. Application Data
(63) Continuation of application No. 12/042,888, filed on Mar. 5, 2008, now abandoned.
(60) Provisional application No. 60/904,995, filed on Mar. 5, 2007.

(51) Int. Cl. A61B 18/04

(52) U.S. Cl. 606/28

(57) ABSTRACT

The advantages of this invention are provided by a surgical tool for repairing a damaged spinal disc of a patient comprising: a catheter having a proximal end and a distal end; a guide wire contained within the catheter so that when the guide wire is pulled, the catheter will flex into a non linear shape allowing it to be steered once inserted in the patient; a heating element carried by the catheter, probe or heating member, and connected to an power source 45 (external or not) so that the heating element can be activated and provide heat to treat a disk; a probe that extends from the catheter so that the probe can be extended out of the catheter; and, a grasping member that can from the distal end of the catheter.

### CLAIM OF PRIORITY

This patent application claims priority on non-provisional patent application Ser. No. 12/042,888 filed Mar. 5, 2008 which in turn claims priority on provisional patent application Ser. No. 60/904,995 filed Mar. 5, 2007 entitled Apparatus For Treating A Damaged Spinal Disk.

### FIELD OF THE INVENTION

The present invention is directed to an apparatus for treating a ruptured, torn, or bulging disc. More specifically, this invention is an apparatus for performing both Percutaneous Microdiscectomy Decompression (PMD) and Intradiscal Electrothermal Annuloplasty (IDET) without the need for multiple insertions of surgical tools into a patient.

### BACKGROUND OF THE INVENTION

Low back pain occurs in about 90% of all people at some time in their lives. One problem in understanding “low back pain” is that it describes a symptom, not a specific disease. It is becoming clear that chronic low back and leg pain is often the result of damage to spinal discs.

The present invention is directed to a medical treatment tool for the treatment of discs in the lumbar, cervical, or thoracic spine that have suffered from a bulge, rupture, or a tear. The disc in the spine absorbs mechanical loads placed on the spine while also allowing the spine to flex. The disc can be damaged due to a number of reasons including aging, degeneration of the disc, or injury to the spine. A herniated disc can be caused by sufficient pressure on the disc by the vertebrae adjacent to it. This could be caused by lifting a heavy object improperly, by sudden twisting, by the weakening of the disc that can occur with age or other reasons, or by traumatic injury to the back.

When the soft interior of the disc bulges out of its normal location, it places pressure on the spinal cord or nerve root. Therefore, pain, nerve damage, or other damage or adverse symptoms can occur. Damage to the disc can result in tears in the outer shell of the disc. The jelly-like material inside the disc, the disc nucleus, may be forced out through the tears or cracks in the outer shell causing a bulge or break.

Damage to the disc can cause pain and it is desirable to treat this pain. Treatment can include strengthening the annulus of the weakened disc wall and to deaden the painful nerves. One option used to treat patients with certain disc pain is Intradiscal Electrothermal Therapy. This involves passing a flexible catheter through a needle into the painful disc. Wires inside the catheter then heat the disc wall resulting in fibrosis which strengthens as well as desensitizes the disc. This requires the catheter to be inserted into the patient through percutaneous approach.

Once inserted, the wire that is inserted into the disc rests beside the area to be heated and therefore treated. Placement of the wire in the disc can be a difficult problem, particularly when the tear in the disc is larger. When the catheter is inserted into the disc, it is desirable to have it travel around the inner circumference of the outer shell. It is undesirable for the catheter to be caught or hung on the tear and even may protrude through the tear. Further, the catheter is difficult to steer. Therefore, it is advantageous to have a catheter which allows a heating wire to be steerable internal to a disc so that the catheter can travel within the disc and steered around the inner part of the disc around to the inner part of the annulus.

Another adverse effect a damaged disc can have is when the disc material within the disc expands to the point that excess pressure is exerted. In the case of a bulging disc, there can be a tear as well as increased pressure in the disc space. The tear can allow for the pressure in the disc to be relieved. When the tear is repaired, there is no place for the pressure to be relieved and the beneficial results of the tear repair may be undermined. In order to relieve the pressure from excess disc material or Percutaneous Microdiscectomy Decompression can be performed to remove material prior to the disc repair. This increases the chances of achieving good results. In the PMD process, the physician removes disc material through a small cannula using a special tool. This tool rotates rapidly and winds disc material around the tool which is then removed along with the wound disc material. Traditionally, this procedure requires the insertion of the special tool into the disc. To perform PMD and IDET together, two surgical tools would have to be inserted into the patient.

With each insertion of a surgical tool, risks associated with the procedure are increased. Therefore, it would be advantageous to provide for a surgical tool that includes the ability to perform PMD and IDET without having to insert two surgical tools or which only require a single tool insertion.

### SUMMARY OF THE INVENTION

The advantages of this invention are provided by a surgical tool for repairing a damaged spinal disc of a patient comprising: a catheter having a proximal end and a distal end; a guide wire contained within the catheter having a first end extending outward from the proximal end of the catheter and a second end carried by the distal end of the catheter so that when the first end is pulled, the catheter will flex into a non linear shape allowing it to be steered once inserted in the patient; a heating element carried by the distal end of the catheter and connected to a power source so that the heating element can be activated and provide heat at the distal end of the catheter so that damage to a disc can be treated with heat when the catheter is inserted in the patient; a probe contained within the catheter wherein the probe extends outward from the proximal end of the catheter so that a distal end of the probe can be pulled into or extended out of the distal end of the catheter; and, a grasping member carried by the distal end of the probe so that when the probe is extended from the distal end of the catheter, the grasping member grabs disc material contained with the spinal disc so that that the disc material can be removed from the spinal disc when the probe is removed from the patient. The heating member can be carried by the catheter, the probe or a separate heating probe.

### DESCRIPTION OF THE DRAWINGS

The invention will be more readily understood by referring to the following drawings and specification.

FIG. 1 is a cross section of a disc showing the invention inserted in the disc;

FIG. 2 is a cross section of a disc showing the invention inserted in the disc;

FIG. 3 is a cross section of a disc showing the invention inserted in the disc;

FIG. 4 is a side view of the invention;

FIG. 5 is a side view of the invention;

FIG. 6 is a perspective view of the invention;

FIG. 7 is a perspective view of the invention;

FIG. 7A is a perspective view of a portion of the invention;

FIG. 7B is a perspective view of a portion of the invention;

FIG. 8 is a perspective view of the invention;

FIG. 9 is a perspective view of the invention;

FIG. 9A is a prospective view of a portion of the invention; and,

FIG. 9B is a perspective view of a portion of the invention.

### DESCRIPTION OF THE INVENTION

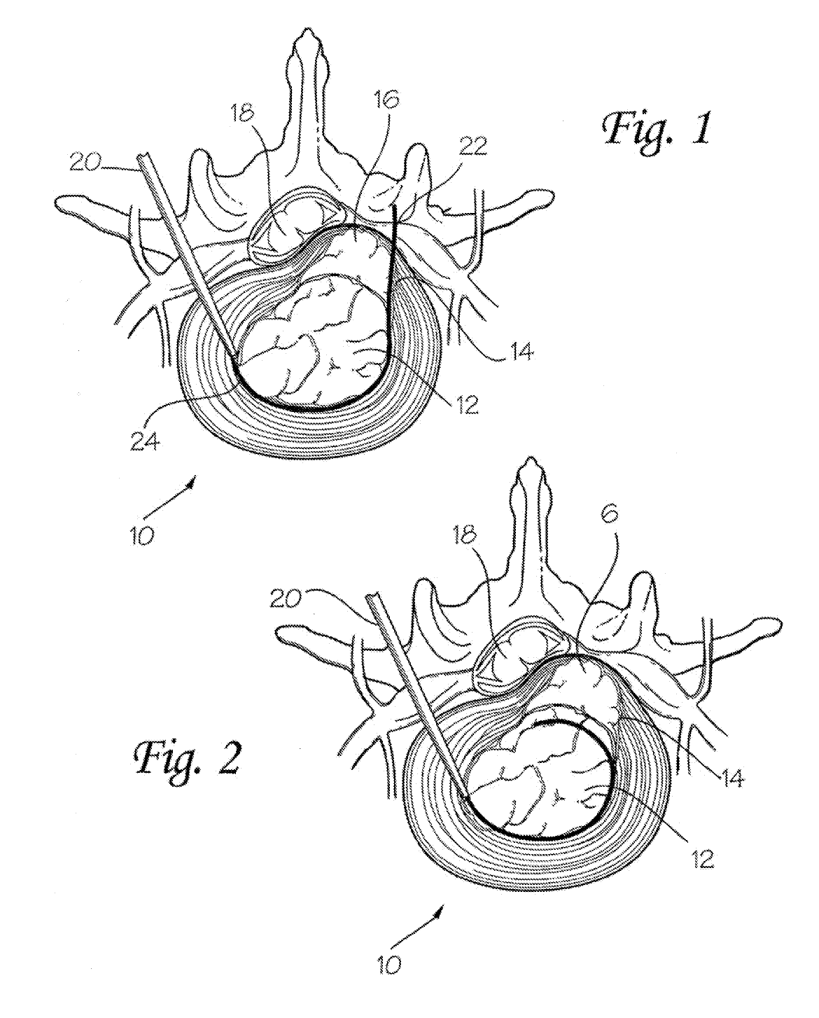

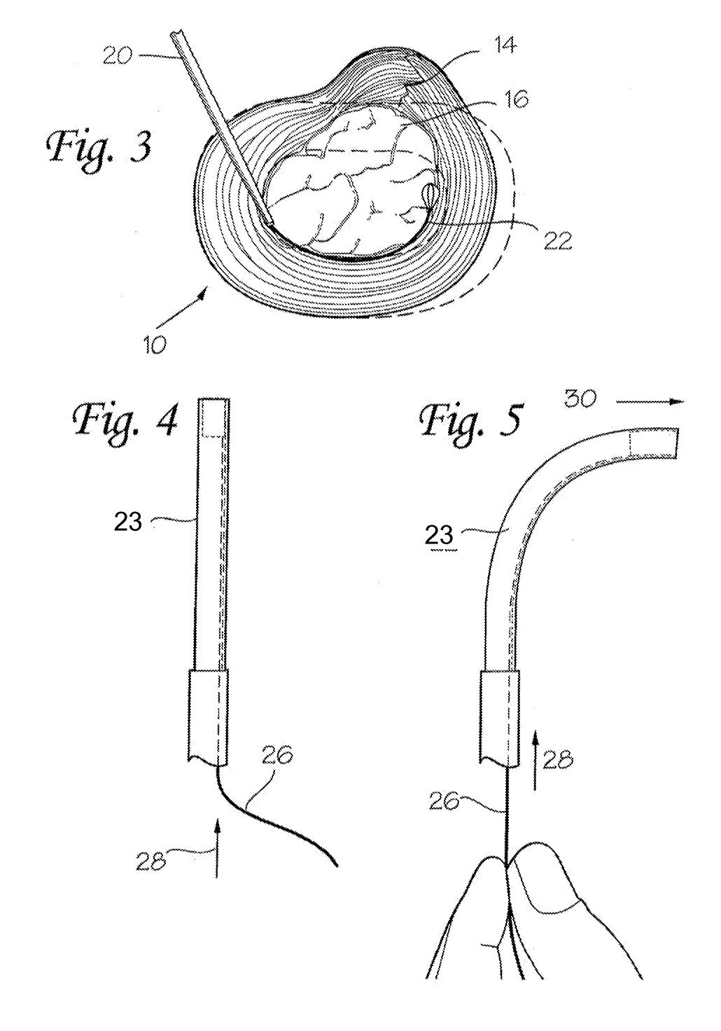

Referring now to FIG. 1, a disc 10 is shown having internal disc material 12. In FIG. 1, a tear 14 has allowed disc material to bulge into area 16. Due to the displacement of the disc material, pressure is placed on spinal cord 18 which can result in pain to the individual. When performing IDET to repair the tear, a surgical tool 20 having a probe 22 is inserted into the disc at location 24. The probe of the surgical tool can be inserted in an area away from the tear so as not to worsen the tear.

In one embodiment, a catheter in included in the tool 20 for surrounding the probe and assisting with the insertion of the probe into the patient. The catheter is inserted into the patient and the probe can extend out an opening defined in the catheter and into the patient. In this embodiment, the catheter is inserted at location 24 and the probe can extend outside the catheter and travel around the circumference of the disc until it reaches the area of tear 14. Once the probe reaches this area, a heating section attached to the probe near the distal end of the probe can be actuated to apply heat to the tear to promote the healing of the tear.

In one embodiment, the catheter I inserted into the patient and travels along the circumference of the damaged disc area, it is important that the catheter (and probe) not extend through the tear as shown in FIG. 1, but to continue to travel around the disc as shown in FIG. 2. Therefore, a guide wire 26 is attached internally to catheter 23 shown in FIGS. 4 and 5. Catheter 23 is flexible so that when the guide wire is pushed in direction shown as 28, catheter 23 flexes so that the tip of the catheter moves in a direction shown as 30 in FIG. 5. When guide wire 26 is pulled, the catheter can return to more of a linear configuration.

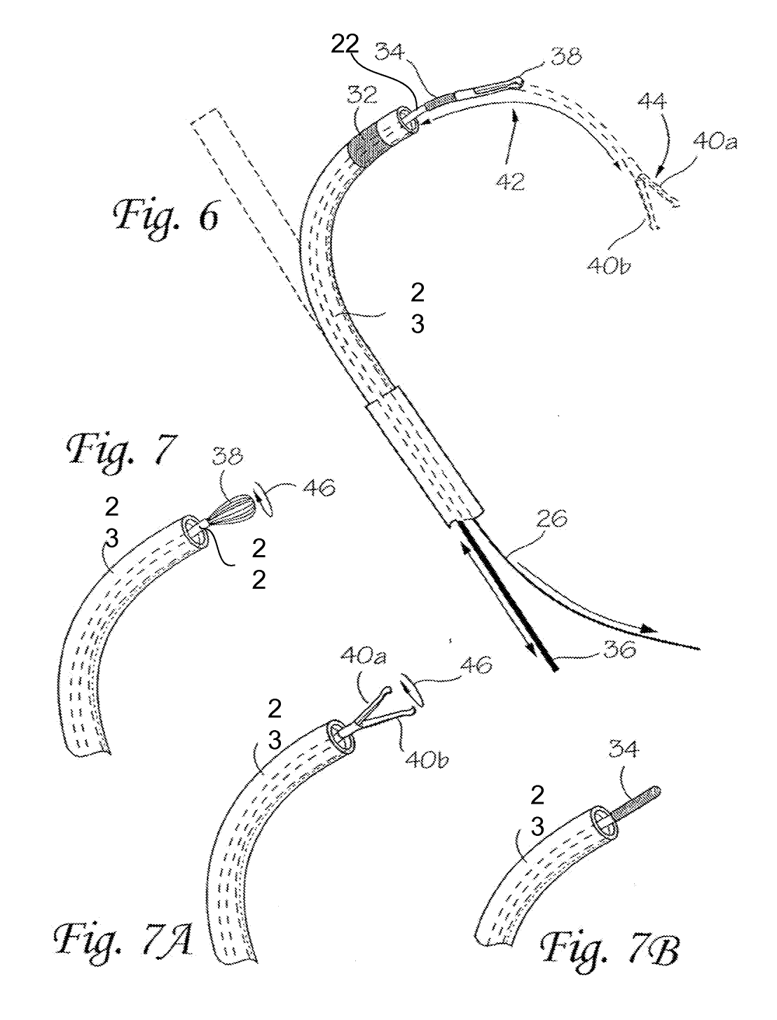

When the catheter is placed within the disc and in the tear location, the tear can be repaired by use of heat therapy as in IDET. In one embodiment, shown in FIG. 6, catheter 23 has an integrated heating element 32. The catheter heating element 34 can then be used to heat areas of the tear to promote healing of the tear. When the heating element is actuated, the heat from the catheter can cause the material around the tear to “heal”, therefore assisting in repairing the tear. In one embodiment, the probe includes a heating element 34 which can also be used to assist the applying heat to the tear to facilitate healing.

It should also be noted that in one embodiment, tool 20 need not include a probe, but just the catheter. In one embodiment, the tool need not include a catheter but just a probe. The probe, in this embodiment, can be inserted directed in to the annular space.

In one embodiment, the probe 22 is steerable within the catheter or the patient. The probe can be biased into a curved arrangement and therefore when rotated about an axis running along the probes, can be steered into a particular direction. For example, rotating the probe in one direction would orientate the probe to curve upwards, while rotating the probe 180° could cause the probe to orientate in the opposite direction. In one embodiment, the catheter is hollow and can receive the probe or other medical devices such as optical fibers, cameras, graspers, heating elements, cutting tools and the like.

Catheter 23 can be hollow allowing for other surgical tools to pass through probe 22. For example, probe 36 can be inserted into an opening on the handle of surgical tool 20 and extend out of catheter 23. The probe can include a heating element 34 which can be used to perform IDET.

The probe can include a grasping member 38. In performing PMD, the disc material can be removed using the grasping member to remove disc material from the disc so that pressure is relieved and the tear can be repaired. Otherwise, the expanded disc material can cause another tear or impede the repair of the existing tear. Therefore, the probe can be inserted into the catheter and therefore into the disc, the grasping member can grasp disc material for removal. Then, the heating element can be actuated to repair the tear without having to insert multiple surgical tools into the patient. In one embodiment, the grasping member is or a whisk configuration as shown as 38 so that when it rotates, it can grasp disc material that can be removed from the disc.

Probe 22 can include a grasping member 38 having a pair of arms 40a and 40b that have a closed position shown as 42 and an open position shown as 44. In the closed positions, the grasping member can travel through the catheter. Once the grasping member clears the end of probe 22, the arms can be placed in the open position to grasp disc material and closed to hold onto the disc material. The probe can then be rotated in a direction shown as 46 in FIG. 7A to wind the disc material around the probe so that the disc material can then be pulled through the probe 22 and therefore removed from the disc.

It should be noted that the grasping member can be removed from the patient through catheter 22 without the catheter being removed from the patient. Therefore, a separate heating probe 34 can be inserted into catheter so that heating probe can be used to treat the damaged disc. Therefore, the grasping member and heating member need not be incorporated in one probe, but can be separate members for insertion into catheter.

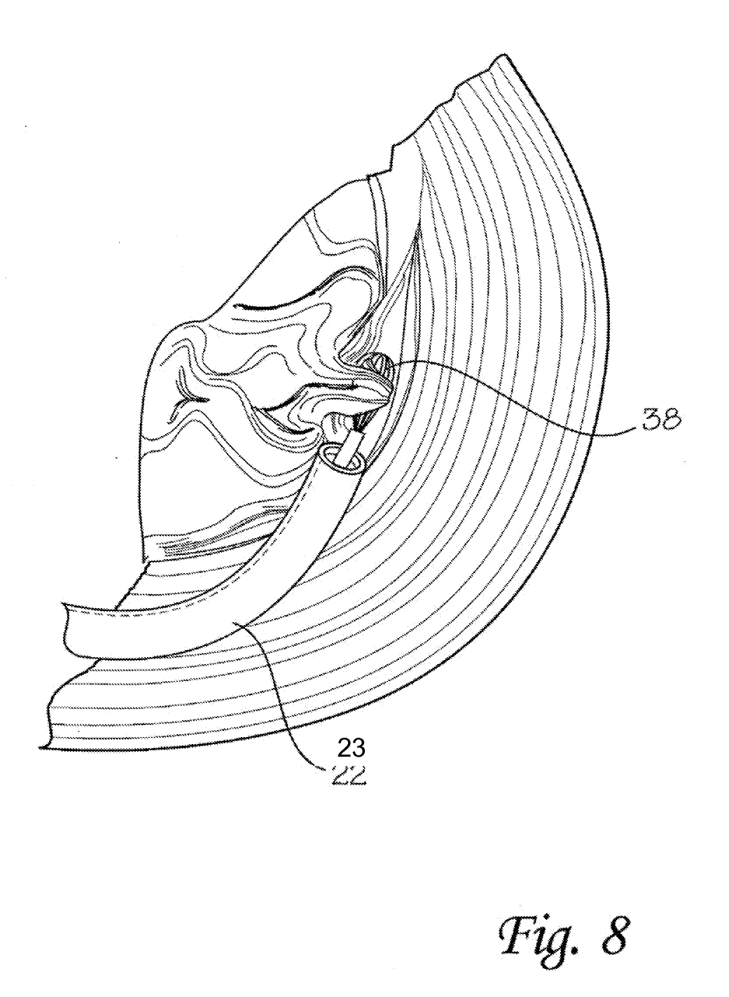

Referring to FIG. 8, once the gasping member whisk is inserted into the disc, the grasping member can be rotated and therefore grasp disc material and pull the disc material from the disc through probe 22.

In one use of the invention, the catheter is inserted into the patient and the disc. The distal end of the catheter is steered in proximity to the tear or location of the disc to be repaired. The probe is extended outward from the distal end of the catheter and the heating element is actuated to assist in repairing the damaged disc. The probe can also be used to grasp disc material with the grasping member and rotating the probe winds disc material around the grasping member and the probe. The catheter can then be removed from the patient and the disc material within. Further, the probe can be retracted within the catheter prior to removing the catheter.

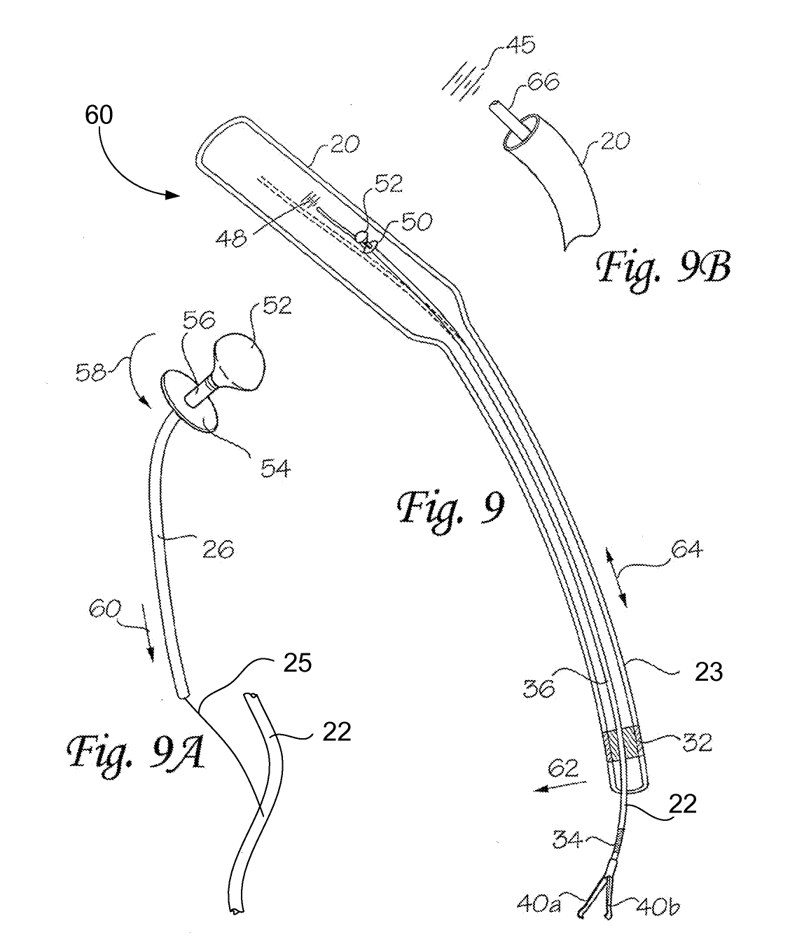

Referring now to FIG. 9, another embodiment is shown. The surgical tool can include a handle 60. The handle can be a housing for allowing the user to hold the surgical device as well as to house various components of the invention. In one embodiment, the handle includes actuating member 52, post 56 and carrier plate 54. In one embodiment, the handle is an elongated housing attached to a proximal end of surgical tool 20. In one embodiment, the housing defines an opening allowing for the insertion of probes and the like. In one embodiment, actuating member is grasping actuating member and in one embodiment, actuating member is the guide wire actuating member.

Surgical tool 20 is shown having probe guide wire 25 contained within the surgical tool and carried by the probe 22. The probe guide wire can include a guide wire actuating member 52 attached to the guide wire which can cause the guide wire to travel within catheter 23. In one embodiment, actuating member 52 is attached to the probe and used to steer the probe. When member 52 (FIG. 9A) is rotated in a direction shown as 58 the probe or catheter guide wire to move in a direction shown as 60. When guide wire moves in direction 60, the distal end of the probe or catheter moves in a direction shown as 62 allowing the probe or catheter to be steered. Therefore, the probe can be steered once inserted in the patient when the distal end of the probe is extending out of the catheter. A carrier plat 54 can be carried by the actuating member.

Probe 36 can also be connected to a probe actuating member 50 attached to the handle. Probe actuating member 50 can cause probe 36 to travel the directions shown as 64 thereby allowing the distal end of probe 36 to extend beyond the distal end of probe 36 and retract within the probe. Power source 48 (FIG. 9B) can be contained within surgical tool 20, such as in the handle, or can be located outside of the surgical tool and be connected to the surgical tool by electrical connection 66. In one embodiment, the structure of the probe actuating member can be used in conjunction with the guide wire of the catheter so that an actuating member can be used to actuate the guide wire and therefore flex the catheter so that the catheter can also be steered.

While the invention has been described using specific terms, such description is for illustrative purposes only and it is to be understood that changes and variations may be made without departing from the spirit or scope of the following claims.

What is claimed is:

1. A surgical tool for repairing a damaged spinal disc of a patient comprising:
a catheter having a proximal end and a distal end;
a guide wire contained within said catheter having a first end extending outward from said proximal end of said catheter and a second end carried by said distal end of said catheter so that when said first end is pulled, said catheter will flex into a non linear shape allowing it to be steered once inserted in the patient;
a heating element carried by said distal end of said catheter and connected to a power source so that said heating element can be activated and provide heat at said distal end of said catheter so that damage to a disc can be treated with heat when said catheter is inserted in the patient;
a probe contained within said catheter wherein said probe extends outward from said proximal end of said catheter so that a distal end of said probe can be pulled into or extended out of said distal end of said catheter; and,
a grasping member carried by said distal end of said probe having a whisk configuration so that when said probe is extended from said distal end of said catheter, said grasping member grabs disc material contained with the spinal disc so that that the disc material can be removed from the spinal disc when said probe is removed from the patient.

2. The apparatus of claim 1 wherein said grasping member includes a first arm and a second arm having an open and a closed position so that said arms will grab disc material when actuated from said open to said closed position.

3. The apparatus of claim 1 wherein said grasping member includes a whisk so that when said grasping member is rotated, said whisk will grab disc material.

4. The apparatus of claim 1 including:
a handle carried by said proximal end of said catheter; and,
a power source carried by said handle and connected to said heating element to provide power to said heating element so that heat can be applied to a damaged disc.

5. The apparatus of claim 1 including:
a handle carried by said proximate end of said catheter; and,
a guide wire actuating member carried by said handle and attached to said first end of said guide wire so that when said guide wire actuating member is actuated, said guide wire is repositioned within said catheter causing said catheter to flex so that said catheter can be steered while being inserted into the patient.

6. The apparatus of claim 1 including:
a handle carried by said proximate end of said catheter; and,
a grasping actuating member carried by said proximal end of said catheter so that when said grasping actuating member is actuated, said catheter can be manipulated before or after insertion into the patient.

7. The apparatus of claim 1 including a probe guide wire contained within said probe having a distal end and connected to said distal end of said probe so that when said probe guide wire is actuated, said probe can be steered.

8. The apparatus of claim 7 including:
a handle carried by said proximate end of said catheter; and,
a grasping actuating member carried by said proximal end of said probe guide wire so that when said grasping actuating member is actuated, said probe guide wire is inserted into the patient and can be steered.

9. A surgical tool for repairing a damaged spinal disc of a patient comprising:
a catheter having a proximal end and a distal end;
a heating element for insertion into said proximal end of said catheter for extending out of said distal end of said catheter and connected to a power source so that said heating element can be activated and provide heat at said distal end of said catheter so that damage to a disc can be treated with heat when said catheter is inserted in the patient;
a probe for being inserted into said proximal end of said catheter for extending out of said distal end of said catheter; and,
a grasping member carried by said distal end of said probe so that when said probe is extended from said distal end of said catheter, said grasping member grabs disc material contained with the spinal disc so that disc material can be removed from the spinal disc when said probe is removed from the patient.

10. The apparatus of claim 9 wherein said grasping member includes a first arm and a second arm having an open and a closed position so that said arms will grab disc material when actuated from said open to said closed position.

11. The apparatus of claim 9 wherein said grasping member includes a whisk so that when said grasping member is rotated, said whisk will grab disc material.

12. The apparatus of claim 9 including a probe guide wire contained within said probe having a distal end and connected to said distal end of said probe so that when said probe guide wire is actuated, said probe can be steered.

13. The apparatus of claim 9 including a probe guide wire contained within said heating element having a distal end and connected to a distal end of said heating element so that when said probe guide wire is actuated, said heating element can be steered.

14. A surgical tool for repairing a damaged spinal disc of a patient comprising:
a catheter having a proximal end and a distal end;
a probe for being inserted into said proximal end of said catheter for extending out of said distal end of said catheter;
a grasping member carried by said distal end of said probe so that when said probe is extended from said distal end of said catheter, said grasping member is rotated to capture disc material contained with a damaged disc that can be removed from the damaged disc when said probe is removed from the patient;
a heating element carried by said distal end of said grasping member and connected to a power source so that said heating element can be activated and provide heat at said distal end of said grasping member so that disc material ca be removed and damage to a disc can be treated with heat when said grasping member is inserted into the spinal disc of the patient.

15. The apparatus of claim 14 wherein said grasping member includes a first arm and a second arm having an open and a closed position so that said arms will grab disc material when actuated from said open to said closed position.

16. The apparatus of claim 14 wherein said grasping member includes a whisk so that when said grasping member is rotated, said whisk will grab disc material.

17. The apparatus of claim 14 including:
a handle carried by said proximal end of said catheter; and,
a power source carried by said handle and connected to said heating element to provide power to said heating element so that heat can be applied to a damaged disc.

18. The apparatus of claim 14 including a guide wire contained within said catheter having a first end extending outward from said proximal end of said catheter and a second end carried by said distal end of said catheter so that when said first end is pulled, said catheter will flex into a non linear shape allowing it to be steered once inserted into a patient.

19. The apparatus of claim 14 including a probe guide wire contained within said probe having a first end extending outward from said proximal end of said probe and a second end carried by said distal end of said probe so that when said first end is pulled, said probe will flex into a non linear shape allowing it to be steered once inserted into a patient.
